(19) United States
(12) Patent Application Publication
(10) Pub. No.: US 2012/0318253 A1
(43) Pub. Date: Dec. 20, 2012

(54) POROUS FILM FOR BAG-CONSTITUTING MEMBER AND BAG-CONSTITUTING MEMBER FOR DISPOSABLE BODY WARMER

(75) Inventors: Hiroshi Nagami, Matsuyama-shi (JP); Yasuhiro Takeda, Matsuyama-shi (JP)

(73) Assignee: NITTO LIFETEC CORPORATION, Matsuyama-shi (JP)

(21) Appl. No.: 13/486,211

(22) Filed: Jun. 1, 2012

(30) Foreign Application Priority Data
Jun. 17, 2011 (JP) 2011-135713

(51) Int. Cl. C08L 23/06; B65D 30/04; A61F 7/08

(52) U.S. Cl. 126/204; 383/105; 521/134

(57) ABSTRACT

Disclosed is a porous film for a bag-constituting member prepared by stretching an unstretched film to be porous. The unstretched film is made from a material composition essentially including a linear low-density polyethylene; an olefinic copolymer having a Vicat softening point of from 20° C. to 50° C. and a density of less than 0.900 g/cm³; and an inorganic filler. The porous film excels in low-temperature heat sealability and gives a bag-constituting member for a body warmer.

### TECHNICAL FIELD

The present invention relates to a porous film (a void film) for a bag-constituting member, and a bag-constituting member for a body warmer, including the porous film.

### BACKGROUND ART

Porous films are now widely used typically in bag-constituting members for enclosing or housing heaters of disposable body warmers; and in bag-constituting members for housing dehumidifiers or deodorants (see, for example, Japanese Unexamined Patent Application Publication (JP-A) No. H11-19113 (PTL 1) and Japanese Unexamined Patent Application Publication (JP-A) No. 2002-36471 (PTL 2)).

The disposable body warmers have structures such as one illustrated in FIG. 4. Specifically, the structure is formed by heat-sealing two bag-constituting members to form a bag, and enclosing or housing a heater 3 in the bag. The two bag-constituting members are a front member 6 and a back member 7. The heater 3 contains, for example, an iron powder as a principal component. A gas-permeable member including a composite member (laminated member; assemblage) typically of a porous film and a nonwoven fabric is used as at least one of the bag-constituting members (generally as the front member 6), for satisfactorily feeding oxygen to the heater.

The porous film should have a satisfactory heat-seal strength. An example of the porous films is a porous film including an ultrahigh-molecular-weight polyethylene, a linear low-density polyethylene, an ethylene-α-olefin copolymer having a density of less than 0.90 g/cm³, and an inorganic filler as essential components. This porous film excels in heat-seal strength upon heat sealing at high temperatures (high-temperature heat sealability) (Japanese Unexamined Patent Application Publication (JP-A) No. 2009-184705 (PTL 3)).

Higher and higher production speed has been recently required for improving productivity typically of body warmers. Such high production speed requires heat sealing at a low temperature and/or for a short time and therefore requires a porous film which can give a high heat-seal strength even when heat-sealed under mild (weak) conditions (under heat sealing conditions of a low temperature and/or a short time).

The porous film disclosed in PTL 3, when heat-sealed under mild conditions into a bag, may have an insufficient heat-seal strength (seal strength) and may thereby cause breakage of the bag, thus being difficult to achieve a high-speed production. Specifically, the porous film disclosed in PTL 3 is still insufficient in heat-seal strength upon heat sealing at low temperatures.

### CITATION LIST

### Patent Literature

PTL 1: Japanese Unexamined Patent Application Publication (JP-A) No. H11-19113
PTL 2: Japanese Unexamined Patent Application Publication (JP-A) No. 2002-36471
PTL 3: Japanese Unexamined Patent Application Publication (JP-A) No. 2009-184705

### SUMMARY OF INVENTION

### Technical Problem

Accordingly, an object of the present invention is to provide a porous film for a bag-constituting member, which has satisfactory low-temperature heat sealability. Another object of the present invention is to provide a porous film for a bag-constituting member which is resistant to edge tear even when heat-sealed under severe (intense) conditions. Still another object of the present invention is to provide a bag-constituting member for a body warmer using the porous film. Yet another object of the present invention is to provide a disposable body warmer using the bag-constituting member.

As used herein the term “low-temperature heat sealability” refers to such a property as to give a sufficient heat-seal strength when heat-sealed at a low temperature; and the term “edge tear” refers to such a phenomenon that a film after heat sealing teared at a boundary region (edge region) between a heat-sealed portion and a non-heat-sealed portion.

### Solution to Problem

After intensive investigations to achieve the objects, the present inventors found that a porous film, when made from raw materials (hereinafter also collectively referred to as a “material composition” or a “material mixture”) including at least an olefinic copolymer having a specific Vicat softening point and a specific density, and an inorganic filler, can have satisfactory low-temperature heat sealability. The present invention has been made based on these findings.

Specifically, the present invention provides a porous film for a bag-constituting member, which is formed by stretching an unstretched film to be porous. The unstretched film is made from a material composition including at least a linear low-density polyethylene; an olefinic copolymer having a Vicat softening point of from 20° C. to 50° C. and a density of less than 0.900 g/cm³; and an inorganic filler.

The linear low-density polyethylene preferably has a density of from 0.917 to 0.930 g/cm³.

The material composition preferably further include a lubricant in a content of from 0.1 to 3.0 parts by weight per 100 parts by weight of the linear low-density polyethylene.

The material composition preferably further includes a polyethylene having a weight-average molecular weight of from 20×10⁴ to 250×10⁴ as measured through high-temperature gel permeation chromatography (high-temperature GPC).

The present invention provides, in another aspect, a bag-constituting member for a body warmer, which includes an assemblage (laminate) including the porous film and a nonwoven fabric.

In addition and advantageously, the present invention provides a disposable body warmer which includes the bag-constituting member.

### Advantageous Effects of Invention

The porous film for a bag-constituting member according to the present invention, as having the above configuration, can exhibit satisfactory heat sealability (a high heat-seal strength) even when heat-sealed under relatively mild conditions (e.g., low-temperature conditions). The porous film, when used in a bag-constituting member for a body warmer, allows the bag-constituting member to have a sufficient heat-seal strength even upon high-speed production of a bag, and this improves the productivity of the bag. The porous film, when having a density of the constitutive linear low-density polyethylene of from 0.917 to 0.930 g/cm³, not only has satisfactory low-temperature heat sealability but also has improved thermal stability, and thereby becomes resistant to problems such as edge tear even under conditions where the film receives a certain quantity of heat. The porous film according to the present invention, when further including, in the material composition, a polyethylene having a weight-average molecular weight of from 20×10⁴ to 250×10⁴ as measured through high-temperature GPC, not only has satisfactory low-temperature heat sealability, but also has further improved thermal stability.

These and other objects, features, and advantages of the present invention will be more fully understood from the following description of embodiments with reference to the attached drawings. All numbers are herein assumed to be modified by the term “about.”

### BRIEF DESCRIPTION OF DRAWINGS

FIG. 1 is a schematic cross-sectional view illustrating an embodiment of bag-constituting member for a body warmer, using a porous film according to the present invention;

FIG. 2 is a schematic cross-sectional view illustrating an embodiment of a disposable body warmer including a bag-constituting member using the porous film according to the present invention;

FIG. 3 is a schematic plan view, viewed from above, illustrating the disposable body warmer including the bag-constituting member using the porous film, as an embodiment of the present invention; and

FIG. 4 is a schematic cross-sectional view illustrating a customary disposable body warmer.

### DESCRIPTION OF EMBODIMENTS

### Porous Film

A porous film for a bag-constituting member according to an embodiment of the present invention (hereinafter also simply referred to as “porous film”) is made from a material composition including at least a linear low-density polyethylene (hereinafter also referred to as “LLDPE”), an olefinic copolymer having a Vicat softening point of from 20° C. to 50° C. and a density of less than 0.900 g/cm³ (hereinafter also referred to as “olefinic copolymer A”), and an inorganic filler. The material composition preferably further includes a lubricant. The material composition also preferably further include a polyethylene having a weight-average molecular weight of 20×10⁴ to 250×10⁴ as measured through high-temperature GPC (hereinafter also referred to as “high-molecular-weight polyethylene”).

The porous film according to the present invention is prepared by stretching an unstretched film to be porous. More specifically, the porous film is prepared by stretching an unstretched film made from the material composition to be porous.

The linear low-density polyethylene is a linear polyethylene being prepared by polymerization of ethylene with an α-olefin monomer having 4 to 8 carbon atoms and having a short-chain branch. The branch preferably has 1 to 6 carbon atoms as its length. Of α-olefin monomers having 4 to 8 carbon atoms for use in the linear low-density polyethylene, preferred are butene-1, hexene-1,4-methyl-pentene-1, and octene-1. The linear low-density polyethylene preferably contains a constitutional unit derived from ethylene in a content of 90 percent by weight or more based on the total amount of constitutional units (constitutional repeating units) derived from (corresponding to) total constitutive monomers. Of such linear low-density polyethylenes, particularly preferred are so-called metallocene-catalyzed linear low-density polyethylenes (metallocene-catalyzed LLDPEs) prepared by using a metallocene catalyst, because they can give more satisfactory heat sealability. Each of different linear low-density polyethylenes may be used alone or in combination.

The linear low-density polyethylene has a density of preferably from 0.917 to 0.930 g/cm³, and more preferably from 0.918 to 0.920 g/cm³. The linear low-density polyethylene, when having a density of 0.917 g/cm³ or more, may help the porous film to have more satisfactory thermal stability and to be more resistant to edge tear upon heat sealing. As used herein the term “density” of a linear low-density polyethylene refers to a density as determined according to International Organization for Standardization (ISO) 1183 (corresponding to Japanese Industrial Standards (JIS) K 7112) standard.

Though not critical, the linear low-density polyethylene has a weight-average molecular weight of preferably less than 30×10⁴, more preferably from 3×10⁴ to 20×10⁴, and furthermore preferably from 5×10⁴ to 6×10⁴, for satisfactory processing suitability (heat sealing suitability). As used herein the term “weight-average molecular weight” refers to a weight-average molecular weight as measured through high-temperature gel permeation chromatography (high-temperature GPC) using a high-temperature GPC system. More specifically, the weight-average molecular weight may be measured, for example, by the following method.

### Measurement Method and Conditions for High-Temperature GPC

A sample is dissolved in o-dichlorobenzene at 140° C. to give a sample solution. The solution is filtrated through a sintered filter having a pore size of 1.0 μm and thereby yields an assay sample. The assay sample is subjected to gel permeation chromatography using a gel permeation chromatograph “Alliance GPC Model 2000” (supplied by Waters Corporation) under the following conditions.

Separation column: Two TSKgel GMH6-HT columns and two TSKgel GMH6-HTL columns (each having an inner diameter of 7.5 mm and a length of 300 mm, supplied by Tosoh Corporation)

Column temperature: 140° C.

Mobile phase: o-Dichlorobenzene

Flow rate: 1.0 ml/minute

Detector: Differential refractive index detector (RI)

Injection volume: 400 μl

Molecular weight calibration: In terms of a polystyrene standard (supplied by Tosoh Corporation)

Though not critical, the linear low-density polyethylene has a melt flow rate (MFR) at 190° C. of preferably from 1.0 to 6.0 (g/10 min), and more preferably from 3.0 to 5.0 (g/10 min), for satisfactory processing suitability. As used herein the term “MFR at 190° C.” of a linear low-density polyethylene refers to a MFR as measured according to ISO 1133 (JIS K 7210) standard under a load of 2.16 kgf.

Though not critical, the linear low-density polyethylene is contained in the material composition in a content of preferably from 30 to 50 percent by weight, and more preferably from 35 to 45 percent by weight, based on the total weight of the material composition, i.e., based on the total weight (100 percent by weight) of the porous film. The range is preferred for good product quality and satisfactory processing suitability.

The porous film includes a LLDPE as a basic ingredient in the material composition and thereby excels in stretchability.

The olefinic copolymer A is not limited and is exemplified by copolymers formed from an α-olefin as an essential monomer component, namely, copolymers having at least a constitutional unit derived from the α-olefin (constitutional unit (constitutional repeating unit) corresponding to the α-olefin). Of such α-olefin monomers, preferred are α-olefin monomers having 2 to 8 carbon atoms, such as ethylene, propylene, butene-1, pentene-1, hexene-1,4-methyl-pentene-1, heptene-1, and octene-1.

The olefinic copolymer A is typically preferably a copolymer having at least a constitutional unit derived from ethylene or propylene. Exemplary olefinic copolymers having at least a constitutional unit derived from ethylene or propylene include ethylene-α-olefin copolymers such as ethylene-propylene copolymers and ethylene-butene copolymers; propylene-α-olefin copolymers such as propylene-butene copolymers; ethylene-vinyl acetate copolymers; ethylene-unsaturated carboxylic acid copolymers such as ethylene-acrylic acid copolymers and ethylene-methacrylic acid copolymers; ethylene-(meth)acrylic ester copolymers such as ethylene-methyl acrylate copolymers, ethylene-ethyl acrylate copolymers, and ethylene-methyl methacrylate copolymers; and ethylene-vinyl alcohol copolymers. Among them, preferred are ethylene-α-olefin copolymers and propylene-α-olefin copolymers, of which copolymers of ethylene or propylene with an α-olefin monomer having 4 to 8 carbon atoms are more preferred, and copolymers of ethylene or propylene with butene-1 are furthermore preferred. These are preferred for more satisfactory heat sealability.

The ethylene-α-olefin copolymer contains a constitutional unit derived from ethylene in a content of preferably from 60 to 95 percent by weight, and more preferably from 80 to 90 percent by weight, based on the total amount of constitutional units derived from total constitutive monomers. The propylene-α-olefin copolymer contains a constitutional unit derived from propylene in a content of preferably from 60 to 95 percent by weight, and more preferably from 80 to 90 percent by weight, based on the total amount of constitutional units derived from total constitutive monomers.

The olefinic copolymer A may further have a constitutional unit derived from another monomer component than olefins or may be a copolymer including only a constitutional unit or units derived from an olefin or olefins. Each of different olefinic copolymers A may be used alone or in combination.

The olefinic copolymer A has a density of less than 0.900 g/cm³, preferably 0.800 g/cm³ or more and less than 0.900 g/cm³, and furthermore preferably from 0.860 to 0.880 g/cm³. The olefinic copolymer A, as having a density of less than 0.900 g/cm³, helps the porous film to have good heat sealability. The olefinic copolymer A, when having a density of 0.800 g/cm³ or more, may help the porous film to have good thermal stability. As used herein the term “density” of the olefinic copolymer A refers to a density as determined according to American Society for Testing and Materials (ASTM) D1505 standard.

The olefinic copolymer A has a weight-average molecular weight of less than 30×10⁴, preferably from 5×10⁴ to 20×10⁴, and more preferably from 8×10⁴ to 15×10⁴, for satisfactory processing suitability and heat sealability.

Though not critical, the olefinic copolymer A has a MFR at 190° C. (MFR(190° C.)) of preferably from 1.0 to 5.0 (g/10 min), and more preferably from 2.0 to 4.0 (g/10 min). As used herein the term “MFR” at 190° C. of the olefinic copolymer A refers to a MFR measured according to ASTM D1238 standard under a load of 2.16 kgf.

The olefinic copolymer A has a Vicat softening point of from 20° C. to 50° C., and preferably from 35° C. to 45° C. An olefinic copolymer having a Vicat softening point of lower than 20° C. may cause the porous film to have insufficient thermal stability to thereby be susceptible to edge tear upon heat sealing. The olefinic copolymer A, as having a Vicat softening point of 50° C. or lower, helps the porous film to have more satisfactory low-temperature heat sealability. The “Vicat softening point” herein refers to a value as measured in accordance with ASTM D1525 standard.

Though not critical, the olefinic copolymer A may be contained in the material composition in a content of typically preferably from 10 to 50 parts by weight, and more preferably from 20 to 40 parts by weight, per 100 parts by weight of the LLDPE. The olefinic copolymer A, when contained in a content of 10 parts by weight or more, helps the porous film to have more satisfactory low-temperature heat sealability. In contrast, the olefinic copolymer A, if contained in a content of more than 50 parts by weight, may cause the unstretched film to be stretched unevenly upon stretching at a low draw ratio or may cause the porous film to have insufficient thermal stability and to suffer from edge tear upon heat sealing.

The porous film, as including the olefinic copolymer A in the material composition, exhibit further higher high heat-seal strength even when heat-sealed at low temperatures.

The high-molecular-weight polyethylene is a polymer formed from ethylene as a principal monomer component, namely, a polymer mainly having a constitutional unit (constitutional repeating unit) derived from ethylene. The high-molecular-weight polyethylene may be either an ethylene single polymer (homopolymer) or a copolymer of ethylene with an α-olefin monomer having 3 to 8 carbon atoms. Among them, an ethylene homopolymer is preferred for satisfactory heat sealability. The high-molecular-weight polyethylene contains a constitutional unit derived from ethylene in a content of preferably from 90 to 100 percent by weight, based on the total amount of constitutional unit(s) derived from total constitutive monomer(s). Each of different high-molecular-weight polyethylenes may be used alone or in combination. The high-molecular-weight polyethylene helps the porous film to be further resistant to edge tear upon high-temperature heat sealing.

The high-molecular-weight polyethylene has a density of preferably from 0.920 to 0.960 g/cm³, and more preferably from 0.930 to 0.955 g/cm³. As used herein the term “density” of the high-molecular-weight polyethylene refers to a density as determined in accordance with ISO 1183 (JIS K 7112) standard.

The high-molecular-weight polyethylene has a weight-average molecular weight (weight-average molecular weight measured through high-temperature GPC) of from 20×10⁴ to 250×10⁴, preferably from 25×10⁴ to 100×10⁴, and more preferably from 30×10⁴ to 80×10⁴. The high-molecular-weight polyethylene, as having a weight-average molecular weight of 20×10⁴ or more, helps the porous film to be more resistant to edge tear upon high-temperature heat sealing. A high-molecular-weight polyethylene having a weight-average molecular weight of more than 250×10⁴ may cause imperfect extrusion or defects (e.g., fisheyes) and may thereby cause the porous film to have poor appearance. The weight-average molecular weight of the high-molecular-weight polyethylene may be measured under the same conditions as in the afore-mentioned high-temperature GPC.

From the viewpoint of heat sealing suitability, the high-molecular-weight polyethylene is preferably one having a broad molecular weight distribution and particularly preferably an ethylene homopolymer having a broad molecular weight distribution. Specifically, the high-molecular-weight polyethylene has a molecular weight distribution in terms of Mw/Mn of from 4.0 to 9.0, and more preferably from 5.0 to 8.0, where Mw represents a weight-average molecular weight; and Mn represents a number-average molecular weight. The Mw/Mn of the high-molecular-weight polyethylene may be measured through GPC and is preferably measured through high-temperature GPC using a high-temperature GPC system. Specifically, the Mw/Mn may be measured under the same conditions as in the aforementioned high-temperature GPC.

The high-molecular-weight polyethylene may be contained in the material composition in a content of 1 part by weight or more, preferably from 1 to 40 parts by weight, more preferably from 5 to 30 parts by weight, and furthermore preferably from 10 to 20 parts by weight, per 100 parts by weight of the LLDPE. The high-molecular-weight polyethylene, when contained in a content of 1 part by weight or more, may help the porous film to be more resistant to edge tear upon high-temperature heat sealing. However, the high-molecular-weight polyethylene, if contained in a content of more than 40 parts by weight, may cause problems such as imperfect extrusion and defects (e.g., fisheyes).

Examples of the inorganic filler include talc, silica, stone powder, zeolite, alumina, aluminum powder, and iron powder; as well as metal carbonates such as calcium carbonate, magnesium carbonate, calcium/magnesium carbonate, and barium carbonate; metal sulfates such as magnesium sulfate and barium sulfate; metal oxides such as zinc oxide, titanium oxide, and magnesium oxide; metal hydroxides such as aluminum hydroxide, magnesium hydroxide, zirconium hydroxide, calcium hydroxide, and barium hydroxide; and metal hydrates (hydrated metallic compounds) such as a hydrate of magnesium oxide and nickel oxide, and a hydrate of magnesium oxide and zinc oxide. Among them, calcium carbonate and barium sulfate are preferred. The inorganic filler plays such a role as to allow voids (pores) to form around the filler as a result of stretching to thereby allow the film to be porous. Though not critical, the inorganic filler may be, for example, tabular or granular in its shape, but is preferably granular (particle-form) so as to form voids (pores) more satisfactorily as a result of stretching. Among them, inorganic particles composed of calcium carbonate are preferred as the inorganic filler. Each of different inorganic fillers may be used alone or in combination.

The inorganic filler (inorganic particles) has a particle size (average particle diameter) of typically preferably from 0.1 to 10 μm, and more preferably from 0.5 to 5 μm, though not critical. An inorganic filler having a particle size of less than 0.1 μm may not satisfactorily contribute to the formation of voids. In contrast, an inorganic filler having a particle size of more than 10 μm may cause breakage during film formation or may cause poor appearance of the film.

Though not critical, the inorganic filler (inorganic particles) may be contained in the material composition in a content of typically preferably from 50 to 150 parts by weight, and more preferably from 100 to 140 parts by weight, per 100 parts by weight of the LLDPE. The inorganic filler, if contained in a content of less than 50 parts by weight, may not satisfactorily contribute to the formation of voids. In contrast, the inorganic filler, if contained in a content of more than 150 parts by weight, may cause breakage during film formation or may cause poor appearance of the film.

Examples of the lubricant (lubricating agent, smoothing agent) include, but are not limited to, stearic acid and stearic acid derivatives. Among them, metal salts of stearic acid, such as aluminum stearate and calcium stearate, are preferred from the viewpoint of stretchability. Each of different lubricants may be used alone or in combination.

Though not critical, the lubricant may be contained in the material composition in a content of typically preferably from 0.1 to 3.0 parts by weight, more preferably from 0.5 to 2.5 parts by weight, furthermore preferably from 1.0 to 2.0 parts by weight, and still more preferably from 1.2 to 1.8 parts by weight, per 100 parts by weight of the LLDPE. The lubricant, if contained in a content of less than 0.1 part by weight, may not protect the film from problems such as film break and pitting corrosion upon stretching of the unstretched film, thus resulting in poor productivity. In contrast, the lubricant, if contained in a content of more than 3.0 parts by weight, may cause the porous film to have an insufficient heat-seal strength.

The porous film may further include any of additives of various kinds within ranges not adversely affecting the advantageous effects of the present invention. Exemplary additives include colorants, age inhibitors (antioxidants), ultraviolet absorbers, flame retardants, and stabilizers.

The material composition for use in the porous film includes the linear low-density polyethylene, the olefinic copolymer A having a Vicat softening point of from 20° C. to 50° C. and a density of less than 0.900 g/cm³, and the inorganic filler as essential components. The material composition may further include the lubricant, high-molecular-weight polyethylene, and/or additives.

The porous film is prepared by stretching (drawing) an unstretched film to be porous (to be stretched into a porous film). The porous film may for example be prepared by a film formation process using melting state such as T-die process or tubular film process. Of such processes, T-die process is preferred. Specifically, the porous film may be prepared by mixing and dispersing the material components with one another using a twin-screw kneader-extruder to give material pellets; melting and extruding the pellets using a single-screw extruder to give an unstretched film; and stretching the unstretched film uniaxially or biaxially into a porous film. The porous film, when to be a multilayer film, is preferably prepared through coextrusion.

The extrusion temperature in the preparation of the porous film is preferably from 180° C. to 250° C., more preferably from 180° C. to 230° C., and furthermore preferably from 190° C. to 220° C. The haul-off of the unstretched film is performed at a speed of preferably from 5 to 25 m/min and at a haul-off roll temperature (cooling temperature) of preferably from 5° C. to 30° C. and more preferably from 10° C. to 20° C.

The porous film may be prepared by stretching the unstretched film typically uniaxially or biaxially (sequentially biaxially or simultaneously biaxially). The uniaxial or biaxial stretching may be performed according to a known or common stretching procedure such as stretching using a roll or stretching using a tenter. The stretching temperature is preferably from 50° C. to 100° C., and more preferably from 60° C. to 90° C. The draw ratio (in one direction) is preferably from 2 to 5 (times), and more preferably from 3 to 4, for stable formation of a satisfactorily porous film. The draw ratio by area in the biaxial stretching is preferably from 2 to 10, and more preferably from 3 to 7.

The porous film has a thickness of typically preferably from 30 to 150 μm, and more preferably from 50 to 120 μm, though not critical.

The porous film according to the present invention may be used as a constitutive member of a bag-constituting member (member constituting a bag). Above all, the porous film is preferably used as a constitutive member of a bag-constituting member having gas permeability, because the porous film has satisfactory gas permeability and thereby satisfactorily supplies oxygen to a heater.

In production at a high speed (namely, in production where the heat sealing step is performed at a high speed), heat sealing should be performed at a low temperature and/or for a short time. A bag-constituting member using the porous film according to the present invention can exhibit a high heat-seal strength even upon heat sealing under relatively mild conditions (e.g., low-temperature conditions). This is because the olefinic copolymer A constituting the porous film has a low Vicat softening point of from 20° C. to 50° C. The bag-constituting member using the porous film therefore exhibits a satisfactory heat-seal strength and gives a bag with better productivity even when the bag is produced at high speed.

In an industrial heat sealing process, the work to be processed absorbs heat from the heat sealer, and the heat sealing temperature gradually decreases from a preset temperature to a stationary state at a low temperature during operation from the beginning of processing until a stable processing temperature is attained. Accordingly, the quantity of heat applied upon heat sealing is relatively large before the heat sealing temperature reaches the stationary state. In this case and in the case where the production is performed at a relatively low speed, and sufficient quantity of heat is applied upon heat sealing, the porous film may suffer from a problem such as edge tear. In a preferred embodiment to avoid this, the linear low-density polyethylene constituting the porous film has a density of from 0.917 to 0.930 g/cm³. The porous film according to this embodiment can have further higher thermal stability while maintaining satisfactory low-temperature heat sealability and becomes more resistant to problems such as edge tear even under conditions where a certain quantity of heat is applied to the film.

In another preferred embodiment, the porous film further includes the high-molecular-weight polyethylene in the material composition. The porous film according to this embodiment can have further higher thermal stability while maintaining satisfactory low-temperature heat sealability. The porous film can therefore be more resistant to edge tear even when heat-sealed under further severe (more intense) conditions, can undergo heat sealing under a wider range of heat sealing conditions, and thereby contributes to higher productivity of the bag.

Bag-Constituting Member

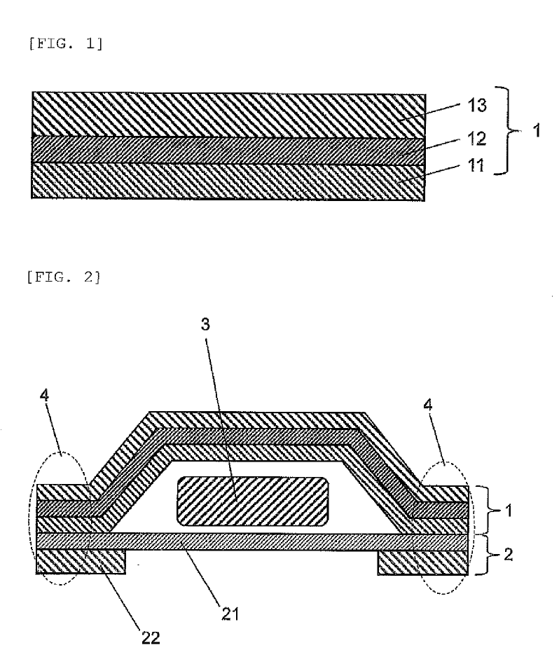

A bag-constituting member may be formed by combining two plies of a porous film according to the present invention or combining (laminating) the porous film with another gas-permeable material than the porous film (hereinafter also referred to as “other gas-permeable material”). In particular, the bag-constituting member is preferably a bag-constituting member including an assemblage including the porous film and a nonwoven fabric (hereinafter also referred to as a “bag-constituting member according to the present invention”) and is more preferably a bag-constituting member including the porous film and a nonwoven fabric layer positioned on or above a surface of the porous film through the medium of an adhesive layer, for satisfactory strength. FIG. 1 is a schematic cross-sectional view illustrating an embodiment of such bag-constituting member using the porous film according to the present invention (bag-constituting member including the porous film). The bag-constituting member 1 includes the porous film 11 (according to the present invention) and a nonwoven fabric layer 13 bonded to each other through the medium of an adhesive layer 12.

Examples of the other gas-permeable material to be combined with the porous film include fibrous materials (e.g., nonwoven fabrics) and other porous films than the porous film according to the present invention. Among them, nonwoven fabrics are preferred as having good feel, smooth texture, and a satisfactory strength.

The nonwoven fabric (nonwoven fabric layer) is not limited and may be any of known or customary nonwoven fabrics including nonwoven fabrics made from natural fibers, and nonwoven fabrics made from synthetic fibers. Examples thereof include polyamide nonwoven fabrics (e.g., nylon nonwoven fabrics), polyester nonwoven fabrics, polyolefin nonwoven fabrics (e.g., polypropylene nonwoven fabrics, polyethylene nonwoven fabrics, and

polypropylene/polyethylene-mixed nonwoven fabrics), and rayon nonwoven fabrics. Among them, nylon nonwoven fabrics (hereinafter also referred to as “nylon-based nonwoven fabrics”, the same is true for others) are preferred, because they have good feel.

The nonwoven fabric may be prepared according to any process and can be, for example, either one prepared by spunbonding (spunbonded nonwoven fabric) or one prepared by spunlacing (spunlace nonwoven fabric). Among them, a spunbonded nonwoven fabric is preferred, for satisfactory strength. The nonwoven fabric may have either a single-layer structure or multilayer structure. The fiber diameter, fiber length, mass per unit area (METSUKE), and other parameters of the nonwoven fabric are not particularly limited. However, the nonwoven fabric preferably has a mass per unit area of from about 20 to 100 g/m² and more preferably from about 20 to 80 g/m², for further satisfactory processability and cost efficiency. The nonwoven fabric may be composed of a fiber of one species or fibers of different species.

In the bag-constituting member, the porous film may be laminated with another gas-permeable material (for example, nonwoven fabric) according to a procedure not limited, but they are preferably bonded with each other through the medium of an adhesive layer, as described above. As used herein the term “adhesive” constituting the adhesive layer also includes and refers to a “pressure-sensitive adhesive (tacky adhesive)”.

The adhesive for constituting the adhesive layer may be any of known adhesives which are exemplified by, but are not limited to, rubber adhesives such as natural rubbers and styrenic elastomers; urethane adhesives (acrylic urethane adhesives); polyolefin adhesives such as ethylene-vinyl acetate copolymers (EVAs) and ethylene-methyl acrylate copolymers (EMAs); acrylic adhesives; silicone adhesives; polyester adhesives; polyamide adhesives; epoxy adhesives; vinyl alkyl ether adhesives; and fluorine-containing adhesives. Among them, polyamide adhesives and polyester adhesives are preferred. Each of different adhesives may be used alone or in combination.

The adhesive for use herein can be an adhesive of every form, but is preferably a hot-melt (thermofusible) adhesive, because it can be applied by heating and melting without the use of solvents, can be directly applied even to a nonwoven fabric to form an adhesive layer, and, when the member is heat-sealed, can give a further higher adhesive strength in the heat-sealed portion. Specifically, the adhesive is preferably a polyamide or polyester hot-melt adhesive and is more preferably a polyamide or polyester thermoplastic hot-melt adhesive.

The specific way to assemble (laminate) the porous film with the other gas-permeable material (particularly nonwoven fabric) may vary depending typically on the type of the adhesive and is not limited. Typically, when a hot-melt adhesive is used, the lamination may be performed by applying the adhesive to the other gas-permeable material (nonwoven fabric) and then affixing the porous film thereto; or by applying the adhesive to the porous film and then affixing the other gas-permeable material (nonwoven fabric) thereto. However, when such a hot-melt adhesive is used, the lamination is preferably carried out by applying the adhesive to the other gas-permeable material (nonwoven fabric), and affixing the porous film thereonto. The application (coating) can be carried out according to any known or customary procedure used for the application of hot-melt adhesives. The application is preferably carried out by spray coating, stripe coating, or dot coating, typically for maintaining the gas permeability. Though not critical, the mass of coating (in terms of solids content) of the adhesive is preferably from 0.5 to 20 g/m² and more preferably from 1 to 8 g/m², from the viewpoints of adhesion of the heat-sealed portion upon bag formation and economical efficiency.

The bag-constituting member is a heat-sealable bag-constituting member which will be heat-sealed to form a bag. The bag-constituting member using the porous film according to the present invention is satisfactorily heat-sealable at a low temperature. The bag has only to include a bag-constituting member using the porous film according to the present invention (preferably the bag-constituting member according to the present invention) at least as part thereof. Specifically, the bag may be formed by heat sealing two or more plies of the bag-constituting members according to the present invention with one another or may be formed by heat sealing a bag-constituting member using the porous film according to the present invention with a bag-constituting member other than the bag-constituting member using the porous film according to the present invention (hereinafter also referred to as “other bag-constituting member”). The resulting bag which has been heat-sealed using the porous film has good low-temperature heat sealability.

The bag-constituting members using the porous film according to the present invention (preferably the bag-constituting members according to the present invention) are applicable to a variety of uses according to the contents to be enclosed or housed in the bag. For example, the bag-constituting members are preferably used in disposable body warmers which house a heater (heating element, exothermic material) as bag-constituting members for body warmers. In addition, the bag-constituting members are also advantageously used for the housing typically of dehumidifiers, deodorants, flavoring agents, and deoxidizers.

Disposable Body Warmer

A bag-constituting member using the porous film according to the present invention (preferably a bag-constituting member according to the present invention), when used, gives a disposable body warmer having at least the bag-constituting member. More specifically, such a disposable body warmer can be formed by heat-sealing two plies of a bag-constituting member using the porous film according to the present invention (particularly two plies of the bag-constituting member according to the present invention) with each other, or heat-sealing the bag-constituting member with the other bag-constituting member, to form a bag; and enclosing or housing a heater (heat generator) in the bag.

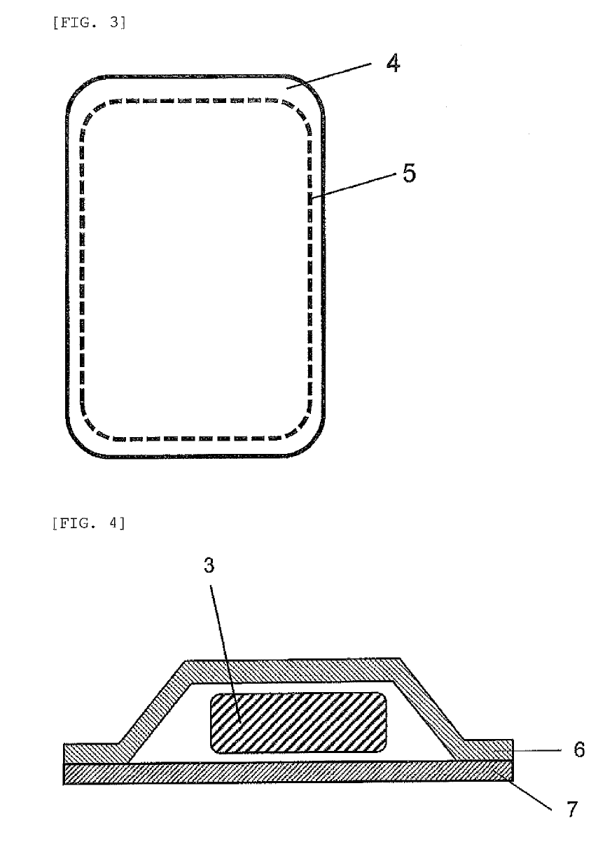

FIGS. 2 and 3 are a schematic cross-sectional view and a schematic plan view viewed from above, respectively, illustrating an embodiment of a disposable body warmer including a bag-constituting member for a body warmer using the porous film according to the present invention. The disposable body warmer illustrated in FIGS. 2 and 3 includes a bag and a heater 3 enclosed in the bag. The bag is produced by heat-sealing the bag-constituting member 1 with another bag-constituting member 2 at an end portion (heat-sealed portion 4). The other bag-constituting member 2 includes a substrate 21 and a pressure-sensitive adhesive layer 22. In such a disposable body warmer including a pressure-sensitive adhesive layer on one side thereof and intended to be applied to an adherend such as clothing as with one illustrated above, the bag-constituting member using the porous film according to the present invention is preferably used at least as a member (so-called front member) opposite to the side to face the adherend, for supplying oxygen to the heater further sufficiently.

The other bag-constituting member is a bag-constituting member other than the bag-constituting member using the porous film according to the present invention and is to be combined with the bag-constituting member using the porous film according to the present invention to form a bag, as mentioned above. The other bag-constituting member is not particularly limited and can be any of known or customary gas-permeable or gas-impermeable bag-constituting members. When used in applications where the bag is applied typically to clothing (e.g., as a disposable body warmer to be applied to a body, clothing, or footwear), the other bag-constituting member is preferably a bag-constituting member having a pressure-sensitive adhesive layer, such as a bag-constituting member including a substrate and a pressure-sensitive adhesive layer (bag-constituting member including at least a substrate and a pressure-sensitive adhesive layer). The bag-constituting member of this type is also available as commercial products such as “Nitotac” supplied by Nitto Lifetec Corporation. “Nitotac” is a pressure-sensitive adhesive sheet for body warmers and is a laminate of a heat-sealable polyolefinic substrate and a styrene-isoprene-styrene block copolymer (SIS) pressure-sensitive adhesive layer.

The substrate preferably includes at least one of a heat-sealable layer, a fibrous layer (for example, nonwoven fabric layer), and a film layer. More specific examples of the substrate include a single layer structure of a heat-sealable layer (heat-sealable film layer); a laminate of a heat-sealable layer (inclusive of heat-sealable film layer) and a fibrous layer; and a laminate of a heat-sealable layer and a non-heat-sealable film layer.

A nonwoven fabric for use in the nonwoven fabric layer may be any of the nonwoven fabrics listed above.

The heat-sealable layer can be formed from a resin having heat sealability (heat-sealable resin) or a heat-sealable resin composition containing such a heat-sealable resin. Though not limited, the heat-sealable resin is preferably a polyolefinic resin (olefinic resin). The polyolefinic resin can be any of resins each containing at least an olefinic component as a monomer component. Examples of the olefinic component include α-olefins such as ethylene, propylene, butene-1, pentene-1, hexene-1,4-methyl-pentene-1, heptene-1, and octene-1. Specific examples of the polyolefinic resin include ethylenic resins such as low-density polyethylenes, linear low-density polyethylenes, high-density polyethylenes, ethylene-vinyl acetate copolymers, and ethylene-α-olefin copolymers (e.g., ethylene-propylene copolymers); propylene resins such as polypropylenes and propylene-α-olefin copolymers; polybutene resins such as poly(butene-1)s; and poly(4-methylpentene-1)s. Exemplary polyolefinic resins usable herein further include copolymers of ethylene and unsaturated carboxylic acids, such as ethylene-acrylic acid copolymers and ethylene-methacrylic acid copolymers; ionomers; copolymers of ethylene and (meth)acrylic esters, such as ethylene-methyl acrylate copolymers, ethylene-ethyl acrylate copolymers, and ethylene-methyl methacrylate copolymers; and ethylene-vinyl alcohol copolymers. Polyethylenes are preferred as the polyolefinic resin for use in the heat-sealable layer, of which low-density polyethylenes, linear low-density polyethylenes, and ethylene-α-olefin copolymers are more preferred. Each of different heat-sealable resins may be used alone or in combination. The heat-sealable layer may have a single-layer structure or multilayer structure.

The α-olefin in the ethylene-α-olefin copolymer for use in the heat-sealable layer is not limited, as long as being an α-olefin other than ethylene, and is typified by α-olefins having 3 to 10 carbon atoms, such as propylene, butene-1, pentene-1, hexene-1,4-methyl-pentene-1, heptene-1, and octene-1. Specific examples of the ethylene-α-olefin copolymer therefore include ethylene-propylene copolymers and ethylene-(butene-1) copolymers. Independently, the α-olefin in the propylene-α-olefin copolymer for use as the olefinic resin in the heat-sealable layer can be chosen as appropriate typically from among α-olefins having 4 to 10 carbon atoms.

Of such heat-sealable resin compositions as mentioned above, preferred are polyolefinic resin compositions each containing at least an ethylene-α-olefin copolymer as an polyolefinic resin, of which polyolefinic resin compositions containing both an ethylene-α-olefin copolymer and at least one of a low-density polyethylene and a linear low-density polyethylene are more preferred. Though not critical, the content of the ethylene-α-olefin copolymer in the polyolefinic resin composition can be chosen within the range of typically 5 percent by weight or more, preferably from 10 to 50 percent by weight, and more preferably from 15 to 40 percent by weight, based on the total weight of the polyolefinic resin. For further satisfactory heat sealability at low temperatures, a low-density polyethylene prepared by the catalysis of a metallocene catalyst is preferred as the linear low-density polyethylene.

The film layer can be any of customary film layers. Exemplary material resins for constituting the film layer include polyester resins and polyolefinic resins. Among them, polyolefinic resins are preferred, because they are available inexpensively and have satisfactory flexibility. Resins as listed in the heat-sealable layer can be used as the polyolefinic resins. The film layer may be either a single-layer film or a multilayer film including two or more layers. Independently, the film layer may be either a non-oriented film, or uniaxially or biaxially oriented film, but is preferably a non-oriented film.

Though not critical, the substrate has a thickness of typically from about 10 to about 500 μm, preferably from about 12 to about 200 μm, and more preferably from about 15 to about 100 μm. The substrate may have undergone one or more treatments, such as backing and antistatic treatments, according to necessity.

The pressure-sensitive adhesive layer provided in the other bag-constituting member has the function of affixing the bag to an adhered upon use. Exemplary pressure-sensitive adhesives for constituting the pressure-sensitive adhesive layer include, but are not limited to, known pressure-sensitive adhesives such as rubber pressure-sensitive adhesives, urethane pressure-sensitive adhesives (acrylic urethane pressure-sensitive adhesives), acrylic pressure-sensitive adhesives, silicone pressure-sensitive adhesives, polyester pressure-sensitive adhesives, polyamide pressure-sensitive adhesives, epoxy pressure-sensitive adhesives, vinyl alkyl ether pressure-sensitive adhesives, and fluorine-containing pressure-sensitive adhesives. Each of different pressure-sensitive adhesives can be used alone or in combination. Among them, rubber and urethane (acrylic urethane) pressure-sensitive adhesives are especially preferred.

Examples of the rubber pressure-sensitive adhesives include rubber pressure-sensitive adhesives each containing any of natural rubbers and synthetic rubbers as a base polymer. Exemplary rubber pressure-sensitive adhesives each containing a synthetic rubber as a base polymer include styrenic rubbers (also called styrenic elastomers) such as styrene-butadiene (SB) rubbers, styrene-isoprene (SI) rubbers, styrene-isoprene-styrene block copolymer (SIS) rubbers, styrene-butadiene-styrene block copolymer (SBS) rubbers, styrene-ethylene-butylene-styrene block copolymer (SEBS) rubbers, styrene-ethylene-propylene-styrene block copolymer (SEPS) rubbers, styrene-isoprene-propylene-styrene block copolymer (SIPS) rubbers, and styrene-ethylene-propylene block copolymer (SEP) rubbers; polyisoprene rubbers; reclaimed rubbers; butyl rubbers (isobutylene-isoprene rubbers); polyisobutylenes; and modified materials derived from these rubbers. Among them, styrenic elastomer pressure-sensitive adhesives are preferred, of which SIS pressure-sensitive adhesives and SBS pressure-sensitive adhesives are more preferred. Each of different rubber pressure-sensitive adhesives may be used alone or in combination.

The urethane pressure-sensitive adhesives can be any known or customary urethane pressure-sensitive adhesives without limitation, but preferred examples thereof include the urethane pressure-sensitive adhesives exemplified in Japanese Patent No. 3860880 and Japanese Unexamined Patent Application Publication (JP-A) No. 2006-288690. Among them, acrylic urethane pressure-sensitive adhesives including isocyanate/polyester polyols are more preferred. Of the acrylic urethane pressure-sensitive adhesives, preferred are expanded or foamed pressure-sensitive adhesives containing bubbles or foams, from the viewpoint of reducing skin irritation when the bag is applied directly to the skin. Such expanded pressure-sensitive adhesives can be prepared, for example, by a process of compounding a known or customary blowing agent into a pressure-sensitive adhesive.

The pressure-sensitive adhesive may be any of pressure-sensitive adhesives of different forms, such as emulsion pressure-sensitive adhesives, solvent-borne pressure-sensitive adhesives, and thermofusible pressure-sensitive adhesives (hot-melt pressure-sensitive adhesives). Among them, thermofusible pressure-sensitive adhesives (hot-melt pressure-sensitive adhesives) are particularly preferred, because they can be directly applied through heating and melting to form pressure-sensitive adhesive layers without using solvents.

The pressure-sensitive adhesive for use herein can be any of pressure-sensitive adhesives of different types (properties), such as pressure-sensitive adhesives having heat curability (heat-curable pressure-sensitive adhesives), in which crosslinks or other structures are formed upon the application of heat, and the adhesives are thereby cured; and pressure-sensitive adhesives having curability by the action of active energy rays (active-energy-ray-curable pressure-sensitive adhesives), in which crosslinks or other structures are formed upon the application of active energy rays, and the adhesives are thereby cured. Among them, active-energy-ray-curable pressure-sensitive adhesives are preferred, because they can be free from solvents and are thereby not excessively impregnated into a nonwoven fabric or porous substrate. The heat-curable pressure-sensitive adhesives may further contain one or more of suitable crosslinking agents and polymerization initiators for exhibiting heat curability for exhibiting curability by heating. The active-energy-ray-curable pressure-sensitive adhesives may further contain one or more of suitable crosslinking agents and photoinitiators for exhibiting curability by the action of active energy rays.

The pressure-sensitive adhesive layer may be protected by a known or customary release film (separator) before use.

A bag-constituting member using the porous film according to the present invention is used to form a bag through heat sealing. The heat sealing may be carried out using any procedure (device), but is preferably carried out through compression bonding with a heat sealer. The heat sealing herein is preferably performed under the following conditions for obtaining both satisfactory sealability and good resistance to edge tear. Specifically, the heat sealing temperature is preferably from 90° C. to 160° C., and more preferably from 90° C. to 130° C. Particularly when the bag-constituting member using the porous film according to the present invention includes a polyolefinic nonwoven fabric, the heat sealing temperature is more preferably from 100° C. to 120° C., and furthermore preferably from 110° C. to 120° C. The heat sealing pressure is preferably from 0.5 to 20 kgf/cm², and more preferably from 2.0 to 20 kgf/cm². The heat sealing time is preferably from 0.001 to 1.0 second, and more preferably from 0.001 to 0.5 second.

In an industrial heat sealing process, production under intense or severe heat sealing conditions has a ceiling in high-speed production (improvement in productivity), because it takes a certain time to perform heat sealing under such severe heat sealing conditions (e.g., heat sealing at a high temperature and/or for a long time). When the production is performed at a high speed (when the heat sealing process is performed at a high speed), heat sealing should be performed at a low temperature and/or for a short time. This requires such a material as to give a satisfactory heat-seal strength even when heat-sealed under mild conditions of a low temperature and/or a short time.

The bag-constituting member using the porous film according to the present invention is satisfactorily heat-sealable at a low temperature and can provide a satisfactory heat-seal strength even when heat-sealed at a relatively low preset temperature. For this reason, the bag-constituting member using the porous film according to the present invention can have a satisfactory heat-seal strength and can give a bag with higher productivity, even when the heat sealing process is performed at a high speed. In a preferred embodiment, a porous film according to the present invention has a density of the linear low-density polyethylene of 0.917 to 0.930 g/cm³. A bag-constituting member using this porous film not only excels in low-temperature heat sealability but also has more satisfactory thermal stability. In another preferred embodiment, a porous film according to the present invention further includes a high-molecular-weight polyethylene in the material composition. A bag-constituting member using this porous film not only excels in low-temperature heat sealability but also is resistant to edge tear even upon heat sealing under severe conditions. The bag-constituting member therefore is satisfactorily heat-sealable under a wide range of conditions and helps the bag to have improved properties such as productivity. In still another preferred embodiment, a porous film according to the present invention has a density of the linear low-density polyethylene of from 0.917 to 0.930 g/cm³ and further includes a high-molecular-weight polyethylene as a raw material. A bag-constituting member using this porous film not only excels in low-temperature heat sealability but also excels in high-temperature heat sealability. This enables heat sealing under a further wider range of conditions and achieves further improved productivity.

As used herein the term “edge tear” refers to such a phenomenon that the bag-constituting member tears at a boundary region 5 between a heat-sealed portion and a non-heat-sealed portion (see FIG. 3).

A disposable body warmer according to the present invention is housed in an outer pouch and is sold as a body warmer product. A base material constituting the outer pouch is not particularly limited and can be any of, for example, plastic base materials; fibrous base materials such as nonwoven fabric base materials and woven fabric base materials, made from fibers of various kinds; and metallic base materials such as metal foil base materials made from metallic components of various kinds. Among them, plastic base materials are preferably used as the base material. Examples of the plastic base materials include polyolefinic base materials such as polypropylene base materials and polyethylene base materials; polyester base materials such as poly(ethylene terephthalate) base materials; styrenic base materials including polystyrene base materials, and styrenic copolymer base materials such as acrylonitrile-butadiene-styrene copolymer base materials; amide resin base materials; and acrylic resin base materials. The base material constituting the outer pouch may have a single layer structure or multilayer structure. Though not critical, the outer pouch preferably has a thickness of from 30 to 300 μm.

In a preferred embodiment, the outer pouch has a layer having gas barrier properties (gas-barrier layer) to inhibit permeation of gaseous components such as oxygen gas and water vapor. The gas-barrier layer is not particularly limited, but is exemplified by oxygen-barrier resin layers such as those made of poly(vinylidene chloride) resins, ethylene-vinyl alcohol copolymers, polyvinyl alcohol)s, and polyamide resins; water-vapor-barrier resin layers such as those made of polyolefins and poly(vinylidene chloride)s; and oxygen-barrier and/or water-vapor-barrier inorganic compound layers, including those made of elementary metals such as aluminum, and those made of metallic compounds including metal oxides such as silicon oxide and aluminum oxide. The gas-barrier layer may be a single layer (e.g., it may be the outer pouch base material itself) or a multilayer laminate.

The outer pouch may be a pouch of any form and structure, such as so-called “four-sided sealed pouch (four side seal pouch),” “three-sided sealed pouch (three side seal pouch),” “pillow style pouch,” “stand-up pouch” (“standing pouch”), or “gusseted pouch.” In a preferred embodiment, the outer pouch is a four-sided sealed pouch. The outer pouch may be prepared using an adhesive, but it is preferably prepared by heat sealing (thermofusing) as typically in a four-sided heat-sealed pouch.

### EXAMPLES

The present invention will be illustrated in further detail with reference to several working examples below. It should be noted, however, that these examples are never construed to limit the scope of the present invention.

Details of linear low-density polyethylenes (metallocene-catalyzed LLDPEs) and ethylene-α-olefin copolymers each prepared using a metallocene catalyst and used in examples and comparative examples below are indicated in Table 1. Blending quantities of material components in material mixtures (material compositions) used in the examples and comparative examples are indicated in Table 2.

### Example 1

A material mixture was prepared by melting and kneading, at 200° C., 100 parts by weight of a linear low-density polyethylene prepared by the catalysis of a metallocene catalyst (metallocene-catalyzed LLDPE (a)), 30 parts by weight of an ethylene-α-olefin copolymer (ethylene-α-olefin copolymer (d)), and 10 parts by weight of a high-molecular-weight ethylene polymer having a weight-average molecular weight of 32×10⁴ (high-molecular-weight ethylene homopolymer) as polymer components with 130 parts by weight of calcium carbonate having an average particle diameter of 1.1 μm (inorganic particles), 0.5 part by weight of calcium stearate, and 0.8 part by weight of an antioxidant. The metallocene-catalyzed LLDPE (a) has a MFR (190° C.) of 3.8 (g/10 min), a density of 0.918 g/cm³, and a Vicat softening point of 101° C. The ethylene-α-olefin copolymer (d) has a MFR (190° C.) of 3.6 (g/10 min), a density of 0.870 g/cm³, and a Vicat softening point of 40° C. The high-molecular-weight ethylene homopolymer has a MFR (190° C.) of 0.08 (g/10 min) and a density of 0.958 g/cm³.

The material mixture was melted and extruded using a single-screw extruder at 210° C. and thereby yielded an unstretched film. Next, the unstretched film was stretched to be porous through uniaxial roll stretching at a stretching temperature of 80° C. in a longitudinal direction (machine direction; MD) to a draw ratio of 4.0 and thereby yielded a porous film 70 μm thick.

Next, an amide hot-melt adhesive was applied to a nylon-based spunbonded nonwoven fabric (having a mass per unit area of 35 g/m³) to a mass of coating of 5 g/m³ through spray coating, to which the porous film was affixed, and thereby yielded a bag-constituting member (gas-permeable bag-constituting member: bag-constituting member using the porous film according to the present invention).

### Example 2

A porous film and a bag-constituting member were prepared by the procedure of Example 1, except for using, instead of the metallocene-catalyzed LLDPE (a), a metallocene-catalyzed LLDPE (b) and using the calcium stearate in an amount of 1.5 parts by weight and the antioxidant in an amount of 1 part by weight, as indicated in Table 2. The metallocene-catalyzed LLDPE (b) has a MFR (190° C.) of 3.8 (g/10 min), a density of 0.924 g/cm³, and a Vicat softening point of 106° C.

### Example 3

A porous film and a bag-constituting member were prepared by the procedure of Example 1, except for using, instead of the high-molecular-weight ethylene copolymer, a high-molecular-weight ethylene-propylene copolymer and using the calcium stearate in an amount of 1.5 parts by weight and the antioxidant in an amount of 1 part by weight, as indicated in Table 2. The high-molecular-weight ethylene-propylene copolymer has a weight-average molecular weight of 79×10⁴, a MFR (190° C.) of 43 (g/10 min), and a density of 0.930 g/cm³.

### Comparative Example 1

A material mixture was prepared by melting and kneading, at 200° C., 100 parts by weight of a metallocene-catalyzed LLDPE (c), 30 parts by weight of an ethylene-α-olefin copolymer (e), and 10 parts by weight of a high-molecular-weight ethylene-propylene copolymer as polymer components with 130 parts by weight of calcium carbonate having an average particle diameter of 1.1 μm (inorganic particles), 1.5 parts by weight of calcium stearate, and 1 part by weight of an antioxidant. The metallocene-catalyzed LLDPE (c) has a weight-average molecular weight of 5×10⁴ to 6×10⁴, a MFR (190° C.) of 2.3 (g/10 min), a density of 0.916 g/cm³, and a Vicat softening point of 93° C. The ethylene-α-olefin copolymer (e) has a weight-average molecular weight of 11×10⁴, a MFR (190° C.) of 3.6 (g/10 min), a density of 0.885 g/cm³, and a Vicat softening point of 55° C. The high-molecular-weight ethylene-propylene copolymer has a weight-average molecular weight of 79×10⁴, a MFR (190° C.) of 43 (g/10 min), and a density of 0.930 g/cm³.

The material mixture was melted and extruded using a single-screw extruder at 210° C. and thereby yielded an unstretched film. Next, the unstretched film was stretched to be porous through uniaxial roll stretching at a stretching temperature of 80° C. in a longitudinal direction (machine direction; MD) to a draw ratio of 4.0 and thereby yielded a porous film 70 μm thick.

Next, an amide hot-melt adhesive was applied to a nylon-based spunbonded nonwoven fabric (having a mass per unit area of 35 g/m³) to a mass of coating of 5 g/m³ through spray coating, to which the porous film was affixed, and thereby yielded a bag-constituting member (gas-permeable bag-constituting member).

### Comparative Example 2

A porous film and a bag-constituting member were prepared by the procedure of Comparative Example 1, except for using, as the polymer components, 100 parts by weight of the metallocene-catalyzed LLDPE (c), 35 parts by weight of the ethylene-α-olefin copolymer (e), and 5 parts by weight of the high-molecular-weight ethylene-propylene copolymer having a weight-average molecular weight of 79×10⁴, as indicated in Table 2.

### Comparative Example 3

A porous film and a bag-constituting member were prepared by the procedure of Comparative Example 1, except for using, as the polymer components, 100 parts by weight of the metallocene-catalyzed LLDPE (c), 20 parts by weight of the ethylene-α-olefin copolymer (e), and 20 parts by weight of a high-molecular-weight ethylene-propylene copolymer having a weight-average molecular weight of 180×10⁴, as indicated in Table 2.

### Comparative Example 4

A porous film and a bag-constituting member were prepared by the procedure of Comparative Example 1, except for using, as the polymer components, 100 parts by weight of the metallocene-catalyzed LIME (c), 20 parts by weight of the ethylene-α-olefin copolymer (e), and 20 parts by weight of a high-molecular-weight ethylene-propylene copolymer having a weight-average molecular weight of 230×10⁴, as indicated in Table 2.

### Comparative Example 5

A porous film and a bag-constituting member were prepared by the procedure of Comparative Example 1, except for using, as the polymer components, 70 parts by weight of the metallocene-catalyzed LIME (c), 10 parts by weight of the ethylene-α-olefin copolymer (e), and 60 parts by weight of the high-molecular-weight ethylene-propylene copolymer having a weight-average molecular weight of 79×10⁴, as indicated in Table 2.

TABLE 1

| | MFR | | Vicat |
|---|---|---|---|
| | (190° C.) | Density | softening point |
| | (g/10 min) | (g/cm³) | (° C.) |
| Metallocene-catalyzed LLDPE (a) | 3.8 | 0.918 | 101 |
| Metallocene-catalyzed LLDPE (b) | 3.8 | 0.924 | 106 |
| Metallocene-catalyzed LLDPE (c) | 2.3 | 0.916 | 93 |
| Ethylene-α-olefin copolymer (d) | 3.6 | 0.870 | 40 |
| Ethylene-α-olefin copolymer (e) | 3.6 | 0.885 | 55 |

TABLE 2

| | | | | | Com. | Com. | Com. | Com. | Com. |
|---|---|---|---|---|---|---|---|---|---|
| | | Example 1 | Example 2 | Example 3 | Ex. 1 | Ex. 2 | Ex. 3 | Ex. 4 | Ex. 5 |
| Material | Metallocene-catalyzed LLDPE (a) | 100 | | 100 | | | | | |
| composition | Metallocene-catalyzed LLDPE (b) | | 100 | | | | | | |
| (part by | Metallocene-catalyzed LLDPE (c) | | | | 100 | 100 | 100 | 100 | 70 |
| weight) | Ethylene-α-olefin copolymer (d) | 30 | 30 | 30 | | | | | |
| | Ethylene-α-olefin copolymer (e) | | | | 30 | 35 | 20 | 20 | 10 |
| | High-molecular-weight ethylene polymer | 10 | 10 | | | | | | |
| | (weight-average molecular weight: 32 × 10⁴) | | | | | | | | |
| | High-molecular-weight ethylene-propylene copolymer | | | 10 | 10 | 5 | | | 60 |
| | (weight-average molecular weight: 79 × 10⁴) | | | | | | | | |
| | High-molecular-weight ethylene-propylene copolymer | | | | | | 20 | | |
| | (weight-average molecular weight: 180 × 10⁴) | | | | | | | | |
| | High-molecular-weight ethylene-propylene copolymer | | | | | | | 20 | |
| | (weight-average molecular weight: 230 × 10⁴) | | | | | | | | |
| | Calcium carbonate | 130 | 130 | 130 | 130 | 130 | 130 | 130 | 130 |
| | Calcium stearate | 0.5 | 1.5 | 1.5 | 1.5 | 1.5 | 1.5 | 1.5 | 1.5 |
| | Antioxidant | 0.8 | 1 | 1 | 1 | 1 | 1 | 1 | 1 |

Evaluations

(1) Appearance of Porous Film

The porous films (after stretching) prepared in the examples and comparative examples were examined on their appearance through visual observation. A sample suffering from neither unmelted undesired substance such as a fisheye nor uneven stretching as a stripe extending in a direction perpendicular to the machine direction of the film was evaluated as having a “good” appearance; and a sample suffering from either of unmelted undesired substance or uneven stretching was evaluated as having a “poor” appearance.

(2) Heat-Seal Strength (130° C.)

As a gas-impermeable bag-constituting member, a pressure-sensitive adhesive sheet for body warmers (“Nitotac” supplied by Nitto Lifetec Corporation) was used. Each of the bag-constituting members (gas-permeable bag-constituting members) prepared in the examples and comparative examples and the gas-impermeable bag-constituting member were respectively dispensed; placed so that the porous film surface of the gas-permeable bag-constituting member face the surface of the base film (surface opposite to the pressure-sensitive adhesive layer) of the gas-impermeable bag-constituting member; and inserted into between two heat-seal rolls.

In this process, the line speed was preset to be 8.5 m/min; and the two heat-seal rolls were respectively heated, in which the temperature of one roll facing the gas-permeable bag-constituting member was preset to be 130° C., and the temperature of the other roll facing the gas-impermeable bag-constituting member was preset to be 140° C. The pressure between the two heat-seal rolls was 720 kPa. Heat sealing, when performed under the above-specified conditions, was defined as heat sealing at a heat sealing temperature of 130° C. Heat sealing of the bag-constituting members was performed under the above-specified conditions and thereby yielded bags (bag materials) heat-sealed at 130° C.

The bags each had dimensions of 130 mm in the machine direction (MD; production line direction) and 95 mm in the cross direction (CD; direction perpendicular to the machine direction), whose four sides were heat-sealed in a width of 5 mm.

The bags were subjected to T-peel tests under conditions mentioned below to measure peel force, in which T-peel was performed between the gas-permeable bag-constituting member (composite member of the porous film and the nonwoven fabric) and the pressure-sensitive adhesive sheet for body warmers (“Nitotac”) as both ends. The peel force herein is a peel force in a heat-sealed portion between the bag-constituting member and the pressure-sensitive adhesive sheet for body warmers (gas-impermeable bag-constituting member). The measured peel force of a sample was defined as a “heat-seal strength (130° C.) (N/15-mm)”.

Apparatus: “Shimadzu Autograph” supplied by Shimadzu Corporation

Sample width: 15 mm

Tensile speed: 300 mm/min

Tensile direction: Machine direction (MD)

Ambient temperature and humidity: 23° C., relative humidity of 50%

Number of repeated tests: n=3

The heat-seal strength of a sample was evaluated according to the following criteria:

10 N/15 mm or more: Excellent

6 N/15 mm or more and less than 10 N/15 mm: Good

4 N/15 mm or more and less than 6 N/15 mm: Somewhat poor

Less than 4 N/15 mm: Poor

(3) Heat-Seal Strength (160° C.)

Bags (bag materials) were prepared, and their peel force was measured by the procedure of above-mentioned “(2) heat-seal strength (130° C.)”, except that the temperature of one of the two rolls facing the gas-permeable bag-constituting member was preset to be 160° C., and the temperature of the other roll facing the gas-impermeable bag-constituting member was preset to be 170° C. Heat sealing, when performed under the above-specified conditions, was defined as heat sealing at a heat sealing temperature of 160° C. Evaluation of the heat-seal strength (160° C.) was performed according to the same criteria as in the “(2) heat-seal strength (130° C.)”.

(4) Edge Tear

Bags were produced through heat sealing at 160° C. by the same procedure as in the “(3) heat-seal strength (160° C.)”. Specifically, 650 bags were produced within about 5 minutes from the beginning of production, from which 50 bags were randomly sampled and subjected to the following evaluation on edge tear using a penetrant.

A cross-shaped cut 10 mm long was made in a central part of the gas-permeable bag-constituting member side of each of the bags so that the cut penetrates only the gas-permeable bag-constituting member, and the nozzle tip of a penetrant spray (product name: a Tanshou Spray Penetrant 2, manufactured by Sumico Lubricant Co., Ltd., supplied by Trusco Nakayama Corporation, component: aromatic hydrocarbon) was inserted into the cut, and the penetrant was sprayed for about 2 seconds. Ten seconds after spraying, the edge of a heat-sealed portion (boundary region between the heat-sealed portion and a non-heat-sealed portion) was visually observed. A sample, in which bleeding of a size (length of the longest portion) of 1 mm or more derived from the penetrant was visually observed, was evaluated as “bag with edge tear”; whereas a sample, in which bleeding of a size (length of the longest portion) of 1 mm or more derived from the penetrant was not visually observed, was evaluated as “bag without edge tear”.

The ratio of the number of bag(s) evaluated as “bag with edge tear” to the number (50) of total bags evaluated is shown in Table 3 as “(Number of edge tear)/(Number of evaluated bags) [(number of bag(s) evaluated as “bag with edge tear)/50]”.

(5) Synthetic Evaluation

Each sample was synthetically evaluated based on the evaluations (1) to (4).

The synthetic evaluation was performed according to the following criteria:

Good: Sample is evaluated as excellent or good in the appearance of the porous film, the heat-seal strength (130° C.), and the heat-seal strength (160° C.) and has a number of bags with edge tear of 5 or less;

Inferior: Sample is evaluated as somewhat poor or poor in at least one of the appearance of the porous film, the heat-seal strength (130° C.), and the heat-seal strength (160° C.), or has a number of bags with edge tear of 6 or more.

The evaluation results are indicated in Table 3.

TABLE 3

| | | Evaluation of bag | | | | | |
|---|---|---|---|---|---|---|---|
| | | | | | | Edge tear | |
| | | | | | | (Number of bags with edge | |
| | Appearance of | Heat-seal strength (130° C.) | | Heat-seal strength (160° C.) | | tear)/(Number of evaluated bags) | Synthetic |
| | porous film | (N/15 mm) | Evaluation | (N/15 mm) | Evaluation | (number/number) | evaluation |
| Example 1 | Good | 10.5 | Excellent | 12.5 | Excellent | 2/50 | Good |
| Example 2 | Good | 8.7 | Good | 11.2 | Excellent | 0/50 | Good |
| Example 3 | Good | 7.5 | Good | 9.0 | Good | 0/50 | Good |
| Com. Ex. 1 | Good | 5.5 | Somewhat poor | 8.0 | Good | 10/50 | Inferior |
| Com. Ex. 2 | Good | 5.8 | Somewhat poor | 10.0 | Excellent | 15/50 | Inferior |
| Com. Ex. 3 | Good | 4.5 | Somewhat poor | 6.7 | Good | 0/50 | Inferior |
| Com. Ex. 4 | Poor | 3.0 | Poor | 5.0 | Somewhat poor | 0/50 | Inferior |
| | (many fisheyes) | | | | | | |
| Com. Ex. 5 | Good | 1.5 | Poor | 3.7 | Poor | 0/50 | Inferior |

As is demonstrated by Table 3, the bags produced by using bag-constituting members including porous films according to the present invention (Examples 1 to 3) each have a satisfactory heat-seal strength and are resistant to edge tear both upon heat sealing at a common temperature of 160° C. and upon heat sealing at a low temperature of 130° C.

In contrast, the bags produced by using bag-constituting members including porous films formed from material compositions each containing an ethylene-α-olefin copolymer having a high Vicat softening point (Comparative Examples 1 to 5) have a insufficient heat-seal strength upon heat sealing at 130° C. Among them, the bags including porous films formed from material compositions each containing a high-molecular-weight ethylene-propylene copolymer having a weight-average molecular weight of 79×10⁴ (Comparative Examples 1 and 2) suffer from edge tear; and the porous film formed from a material composition containing a high-molecular-weight ethylene-propylene copolymer having a weight-average molecular weight of 230×10⁴ (Comparative Example 4) suffers from a large amount of fisheyes.

While preferred embodiments of the present invention have been described using specific terms, such description is for illustrated purposes only, and it is to be understood that various changes and modifications may be made without departing from the spirit and scope of the present invention as defined in the appended claims.

### REFERENCE SIGNS LIST

1 bag-constituting member (gas-permeable bag-constituting member)
11 porous film
12 adhesive layer
13 nonwoven fabric layer
2 other bag-constituting member (gas-impermeable bag-constituting member)
21 substrate
22 pressure-sensitive adhesive layer
3 heater
4 heat-sealed portion
5 boundary region between heat-sealed portion and non-heat-sealed portion
6 bag-constituting member (front member)
7 bag-constituting member (back member)

1. A porous film for a bag-constituting member, the porous film formed by stretching an unstretched film to be porous, the unstretched film made from a material composition comprising:
a linear low-density polyethylene;
an olefinic copolymer having a Vicat softening point of from 20° C. to 50° C. and a density of less than 0.900 g/cm³; and
an inorganic filler as essential components.

2. The porous film for a bag-constituting member, according to claim 1, wherein the linear low-density polyethylene has a density of from 0.917 to 0.930 g/cm³.

3. The porous film for a bag-constituting member, according to claim 1, wherein the material composition further comprises a lubricant in a content of from 0.1 to 3.0 parts by weight per 100 parts by weight of the linear low-density polyethylene.

4. The porous film for a bag-constituting member, according to claim 1, wherein the material composition further comprises a polyethylene having a weight-average molecular weight of from 20×10⁴ to 250×10⁴ as measured through high-temperature gel permeation chromatography (high-temperature GPC).

5. A bag-constituting member for a body warmer, comprising an assemblage including:
the porous film of claim 1; and
a nonwoven fabric.

6. A disposable body warmer comprising the bag-constituting member of claim 5.

7. The porous film for a bag-constituting member, according to claim 2, wherein the material composition further comprises a lubricant in a content of from 0.1 to 3.0 parts by weight per 100 parts by weight of the linear low-density polyethylene.

8. The porous film for a bag-constituting member, according to claim 2, wherein the material composition further comprises a polyethylene having a weight-average molecular weight of from 20×10⁴ to 250×10⁴ as measured through high-temperature gel permeation chromatography (high-temperature GPC).

9. The porous film for a bag-constituting member, according to claim 3, wherein the material composition further comprises a polyethylene having a weight-average molecular weight of from 20×10⁴ to 250×10⁴ as measured through high-temperature gel permeation chromatography (high-temperature GPC).

10. The porous film for a bag-constituting member, according to claim 7, wherein the material composition further comprises a polyethylene having a weight-average molecular weight of from 20×10⁴ to 250×10⁴ as measured through high-temperature gel permeation chromatography (high-temperature GPC).

11. A bag-constituting member for a body warmer, comprising an assemblage including:
the porous film of claim 2; and
a nonwoven fabric.

12. A bag-constituting member for a body warmer, comprising an assemblage including:
the porous film of claim 3; and
a nonwoven fabric.

13. A bag-constituting member for a body warmer, comprising an assemblage including:
the porous film of claim 4; and
a nonwoven fabric.

14. A bag-constituting member for a body warmer, comprising an assemblage including:
the porous film of claim 7; and
a nonwoven fabric.

15. A bag-constituting member for a body warmer, comprising an assemblage including:
the porous film of claim 8; and
a nonwoven fabric.

16. A bag-constituting member for a body warmer, comprising an assemblage including:
the porous film of claim 9; and
a nonwoven fabric.

17. A bag-constituting member for a body warmer, comprising an assemblage including:
the porous film of claim 10; and
a nonwoven fabric.
